(19) United States
(12) Patent Application Publication
(10) Pub. No.: US 2025/0315954 A1
(43) Pub. Date: Oct. 9, 2025

(54) SYSTEMS AND METHODS FOR DETERMINING A POSITION OF AN ELECTRODE LEAD WITHIN A COCHLEA

(71) Applicant: Advanced Bionics LLC, Valencia, CA (US)

(72) Inventors: Stephan Geiger, Ventura, CA (US); Pierre Guillon, Stäfa (CH); Tim Nauwelaers, Hannover (DE); Ersin Avci, Isernhagen (DE)

(21) Appl. No.: 18/992,416

(22) PCT Filed: Jun. 22, 2023
(86) PCT No.: PCT/US2023/025969

§ 371 Date: Jan. 8, 2025

Related U.S. Application Data
(60) Provisional application No. 63/393,707, filed on Jul. 29, 2022.

(51) Int. Cl. G06T 7/00; G06T 5/00; G06T 7/11; G06T 7/136; G06T 7/62; G16H 30/40

(52) U.S. Cl. CPC G06T 7/0014; G06T 5/00; G06T 7/11; G06T 7/136; G06T 7/62; G16H 30/40; G06T 2207/10081; G06T 2207/20072; G06T 2207/20076; G06T 2207/20172; G06T 2207/30052

(57) ABSTRACT

An exemplary system comprises memory that stores instructions; and a processor communicatively coupled to the memory and configured to execute the instructions to perform a process. The process may comprise accessing post-operative scan images of a cochlea after an electrode lead insertion procedure, the post-operative scan images depicting an electrode lead with a plurality of electrode contacts inserted at least partially within the cochlea, processing the post-operative scan images together with an active shape model (ASM) of the cochlea to determine candidate positions of the plurality of electrode contacts in relation to the cochlea, and determining, based on the candidate positions of the plurality of electrode contacts, a position of each electrode contact included in the plurality of electrode contacts in relation to the cochlea.

### RELATED APPLICATIONS

The present application claims priority to U.S. Provisional Patent Application No. 63/393,707, filed Jul. 29, 2022, the contents of which is hereby incorporated by reference in its entirety.

### BACKGROUND INFORMATION

Cochlear implant systems are used to provide, restore, and/or improve the sense of hearing to recipients with severe or profound hearing loss. Conventional cochlear implant systems include various components configured to be implanted within a recipient. For example, an electrode lead may be inserted into a cochlea of the recipient and stimulation current may be applied by electrodes on the electrode lead as directed by a cochlear implant that is also surgically implanted within the recipient.

The insertion of an electrode lead into the cochlea of the recipient is performed by way of a delicate surgical procedure that can result in the electrode lead being mispositioned and/or causing trauma to the cochlea of the patient. As such, a postoperative verification process may be useful to detect mispositioning of the electrode lead, trauma in the inner structure of the cochlea, and/or to provide customized activation based on the position of metallic electrode contacts on the electrode lead. Such a verification process may include imaging the cochlea with a scanning device such as a computerized tomography (CT) scanning device. However, determining the position of the electrode lead in relation to the cochlea from scan images such as CT scan images is typically difficult due to the poor resolution of the scan images and/or artifacts in the scan images caused by the metallic electrode contacts of the electrode lead.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1 during a scanning operation.

FIG. 4 shows an exemplary electrode lead position detection system that may be implemented according to principles described herein.

FIGS. 5-6 show exemplary flow diagrams depicting operations that may be performed according to principles described herein.

FIG. 7 shows an exemplary cochlea model that has been sliced into a plurality of segments for processing according to principles described herein.

FIG. 8 shows an exemplary graph that shows pixel intensities with respect to length of an electrode lead according to principles described herein.

FIG. 9 shows an exemplary diagram depicting an arrangement of blobs that may be represented in scan images according to principles described herein.

FIGS. 10A-10C show exemplary electrode lead graphs that may be used during a graph matching process according to principles described herein.

FIG. 11 shows an exemplary flow diagram depicting operations of a graph matching process that may be performed according to principles described herein.

FIG. 12 shows an exemplary flow diagram depicting operations that may be performed to generate a cochlea model according to principles described herein.

FIG. 13 shows an exemplary method according to principles described herein.

FIG. 14 shows an exemplary computing device that may be implemented according to principles described herein.

### DETAILED DESCRIPTION

Systems and methods for determining the position of an electrode lead within a cochlea are described herein. An exemplary system comprises memory that stores instructions; and a processor communicatively coupled to the memory and configured to execute the instructions to perform a process. The process may comprise accessing post-operative scan images of a cochlea after an electrode lead insertion procedure, the post-operative scan images depicting an electrode lead with a plurality of electrode contacts inserted at least partially within the cochlea, processing the post-operative scan images together with an active shape model (ASM) of the cochlea to determine candidate positions of the plurality of electrode contacts in relation to the cochlea, and determining, based on the candidate positions of the plurality of electrode contacts, a position of each electrode contact included in the plurality of electrode contacts in relation to the cochlea.

The systems and methods described herein may provide various benefits to cochlear implant recipients, as well as others involved with managing cochlear implant systems. For example, the systems and methods such as those described herein may facilitate determining the position of an implanted electrode lead from post-operative scan images (e.g., low resolution CT scan images) of the cochlea. As a result, systems and methods such as those described herein may facilitate determining whether the electrode lead is positioned properly (e.g., at the proper depth, has proper cochlea wall contact, etc.) or is positioned improperly (e.g., not adequately within the cochlea, has translocated the basilar membrane, etc.). In addition, by determining the position of the electrode lead in relation to the cochlea, it may be possible to optimize stimulation programs and/or stimulation parameters implemented by a cochlear implant system for a particular recipient.

Various embodiments will now be described in more detail with reference to the figures. The disclosed systems and methods may provide one or more of the benefits mentioned above and/or various additional and/or alternative benefits that will be made apparent herein.

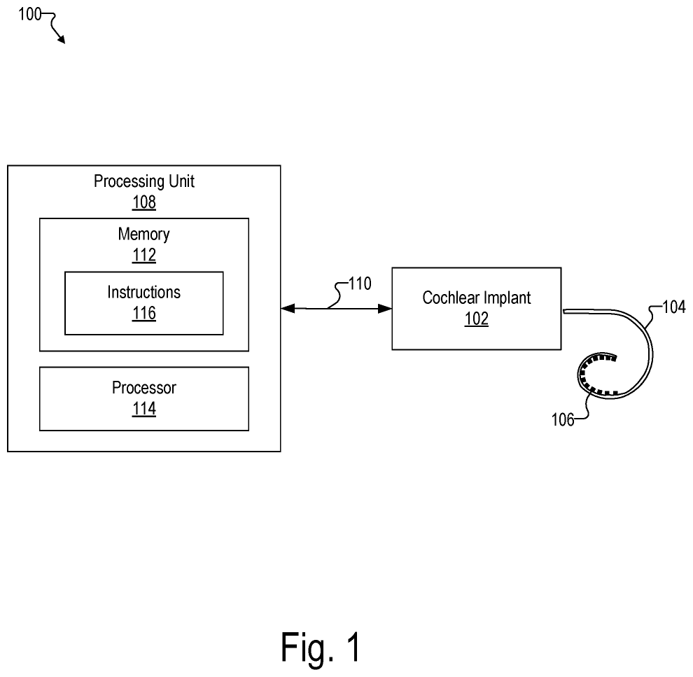

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a brainstem implant and/or any other type of device that may be implanted within the recipient and configured to apply electrical stimulation to one or more stimulation sites located along an auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106. In certain examples, electrode lead 104 may alternatively be referred to as an electrode array.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

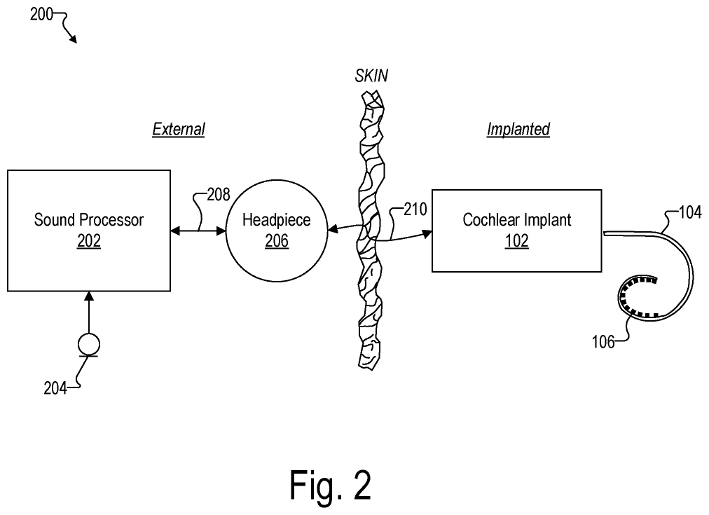

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

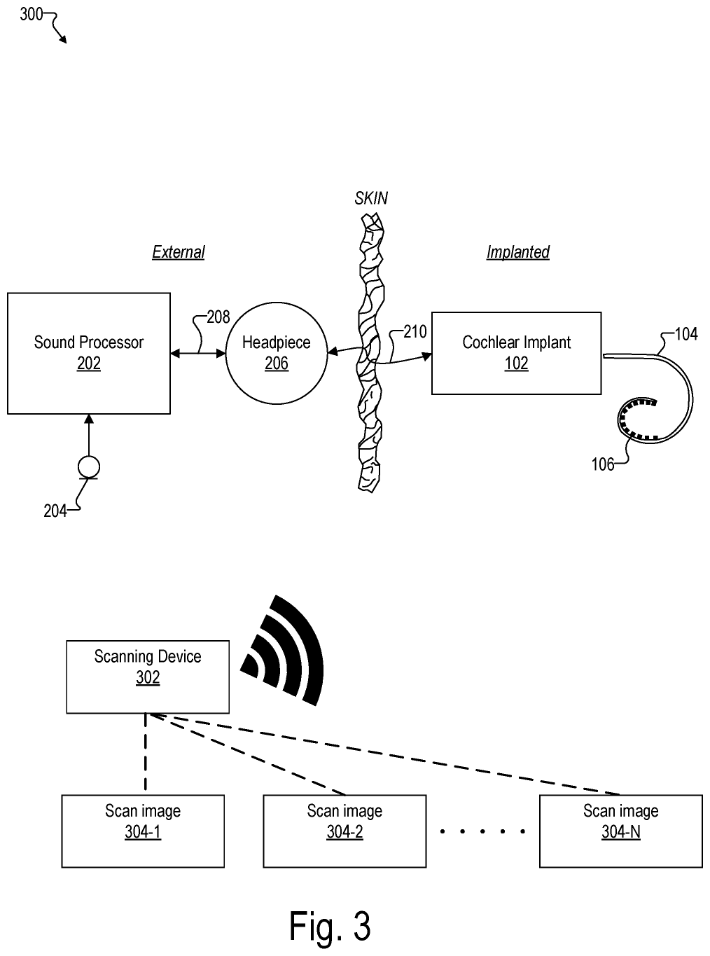

FIG. 3 shows an exemplary implementation 300 that depicts electrode lead 104 of cochlear implant system 100 being imaged by a scanning device 302 after electrode lead 104 has at least partially been inserted within the cochlea.

Scanning device 302 may correspond to any suitable type of scanning device as may serve a particular implementation. For example, scanning device 302 may correspond to a CT scanning device, a magnetic resonance imaging (MRI) device, and/or any other suitable type of scanning device. As shown in FIG. 3, scanning device 302 may be configured to capture or otherwise generate a plurality of post-operative scan images 304 (e.g., post-operative scan images 304-1 through 304-N). In certain examples, scanning device 302 may additionally be used to capture or otherwise generate pre-operative scan images of the cochlea prior to electrode lead 104 being inserted into the cochlea. Scanning device 302 may be configured to capture or otherwise generate any suitable number of pre-operative scan images and/or post-operative scan images as may serve a particular implementation.

Post-operative scan images such as post-operative scan images 304 may show a position of an electrode lead (e.g., electrode lead 104) in relation to the cochlea. However, it is often difficult to identify the electrode contacts (e.g., electrodes 106) on the electrode lead and determine where they are in relation to the cochlea based on the post-operative scan images. For example, post-operative CT scan images are typically low-resolution images where it may be difficult to differentiate potential electrode contact positions from one another. In addition, regions of high intensity (e.g., bright spots) in a scan image may correspond to a position of an electrode contact but may also be caused by anatomical features such as bone structure. Further, additional regions of high intensity may occur due to artifacts caused in the scan images by the metallic electrode contacts of an electrode lead. Accordingly, it is desirable to perform one or more operations such as described herein to process post-operative scan images to facilitate determining the position of the electrode contacts of an electrode lead in relation to the cochlea.

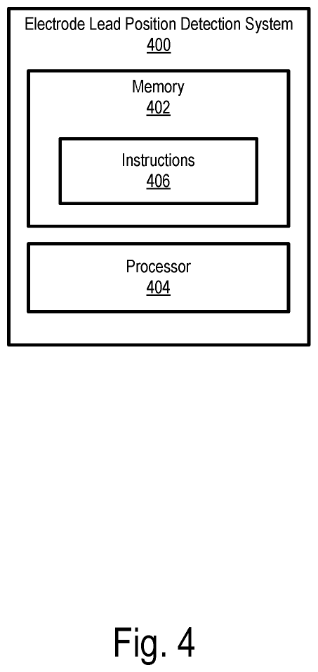

To that end, FIG. 4 shows an exemplary electrode lead position detection system 400 (“system 400”) that may be implemented according to principles described herein. As shown, system 400 may include, without limitation, a memory 402 and a processor 404 selectively and communicatively coupled to one another. Memory 402 and processor 404 may each include or be implemented by hardware and/or software components (e.g., processors, memories, communication interfaces, instructions stored in memory for execution by the processors, etc.). In some examples, memory 402 and/or processor 404 may be implemented by any suitable computing device. In other examples, memory 402 and/or processor 404 may be distributed between multiple devices and/or multiple locations as may serve a particular implementation. Illustrative implementations of system 400 are described herein.

Memory 402 may maintain (e.g., store) executable data used by processor 404 to perform any of the operations described herein. For example, memory 402 may store instructions 406 that may be executed by processor 404 to perform any of the operations described herein. Instructions 406 may be implemented by any suitable application, software, code, and/or other executable data instance.

Memory 402 may also maintain any data received, generated, managed, used, and/or transmitted by processor 404. Memory 402 may store any other suitable data as may serve a particular implementation. For example, memory 402 may store data associated with pre-operative scan images, post-operative scan images, ASM data, volumes of interest data, electrode lead graph data, graph matching parameters, cochlea landmark data, cochlea model database information, 3D cochlea model data, cochlear implant information (e.g., electrode lead dimensions information), graphical user interface content, and/or any other suitable data.

Processor 404 may be configured to perform (e.g., execute instructions 406 stored in memory 402 to perform) various processing operations associated with determining a position of an electrode lead in relation to a cochlea. For example, processor 404 may perform one or more operations described herein to process post-operative scan images together with an ASM of the cochlea to determine candidate positions of electrode contacts of an electrode lead. These and other operations that may be performed by processor 404 are described herein.

System 400 may be implemented in any suitable manner. For example, system 400 may be implemented by any suitable computing device (e.g., a desktop computer, a laptop computer, a cloud computing device, etc.) that may be configured to access and process scan images according to operations such as those described herein. In certain examples, system 400 may include scanning device 302, may be communicatively coupled to scanning device 302, or may otherwise receive, in any suitable manner, pre-operative and/or post-operative scan images of the cochlea captured by scanning device 302.

In some examples, system 400 may be implemented by a computing device that represents a fitting device configured to be selectively used (e.g., by a clinician) to fit sound processor 202 and/or cochlear implant 102 to the recipient. In these examples, the computing device may be configured to execute a fitting program configured to set one or more operating parameters of sound processor 202 and/or cochlear implant 102 to values that are optimized for the recipient. As such, in these examples, the computing device may be considered to be separate from cochlear implant system 100 such that the computing device may be selectively coupled to cochlear implant system 100 when it is desired to fit sound processor 202 and/or cochlear implant 102 to the recipient.

In certain examples, the methods described herein may be performed automatically by system 400. As used herein, the expression “automatically” means that an operation (e.g., an operation determining candidate positions for electrode contacts based on post-operative scan images) or series of operations are performed without requiring further input from a user. For example, system 400 may automatically perform any of the operations described herein based on post-operative scan images without requiring further input from a user.

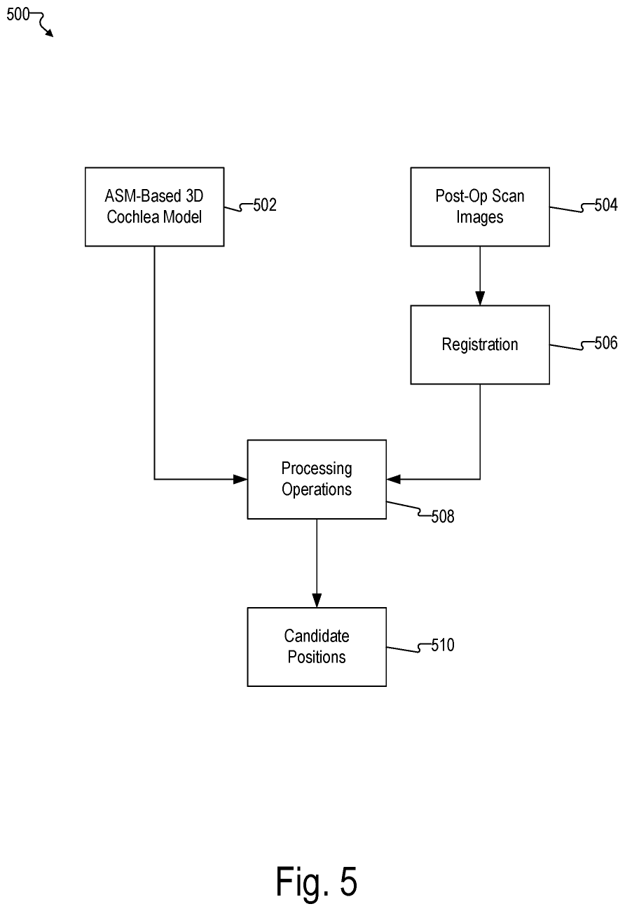

FIG. 5 illustrates an exemplary flow diagram 500 that depicts various operations that may be performed by system 400 to determine a position of an electrode lead in relation to a cochlea. As shown in FIG. 5, system 400 may access an ASM-based 3D cochlea model at operation 502. As used herein, an “active shape model” (ASM) is a statistical shape model (SSM) of a shape (e.g., a cochlea) that deforms to fit an example that shape. An SSM is a geometric model that describes a collection of semantically similar shapes in a compact way and may be composed of an average shape as well as the main modes of shape variations. An ASM may be generated in any suitable manner. For example, an ASM may be generated based on high resolution scan images acquired from temporal bones of donors. In such examples, donors may be used because the high radiation doses associated with high resolution scan images may be too dangerous for a living patient.

The ASM-based 3D cochlea model may represent an ASM that has been deformed to fit one or more determined or estimated surfaces of the cochlea such as a surface of the cochlea wall and/or a surface of the basilar membrane, resulting in an estimation of the anatomical structure of the cochlea. In certain examples, the ASM-based 3D cochlea model may be specific to a particular recipient of a cochlear implant.

In certain examples, the ASM-based 3D cochlea model may be previously generated based on pre-operative scan images (e.g., low resolution pre-operative CT scan images). In such examples, system 400 may access the ASM-based 3D cochlea model in any suitable manner from any suitable source. In certain alternative examples, system 400 may generate the ASM-based 3D cochlea model in any suitable manner based on pre-operative scan images of a recipient and/or post-operative scan images of the recipient. An exemplary process for generating the ASM-based 3D cochlea model is described herein.

At operation 504, system 400 may access post-operative scan images (e.g., post operative scan images 304) of a cochlea after an electrode lead insertion procedure. The post-operative scan images may depict an electrode lead (e.g., electrode lead 104) inserted at least partially within the cochlea. The post-operative scan images may include any suitable number of post-operative scan images as may serve a particular implementation.

At operation 506, system 400 may register the post-operative scan images. This may be accomplished in any suitable manner. For example, system 400 may optimize alignment of the post-operative scan images with the ASM-basd 3D cochlea model based on an intensity of all pixels (e.g., voxels) and a metric (e.g., mutual information between the post-operative scan images and the ASM-based 3D cochlea model). In so doing, regions of high intensity (e.g., bright spots or blobs) in the post-operative scan images that may be indicative of electrode contact positions may be aligned with corresponding positions in the pre-operative scan images and the ASM-based 3D cochlea model.

At operation 508, system 400 may perform a plurality of processing operations to process the post-operative scan images together with the ASM-based 3D cochlea model. System 400 may perform any suitable number of processing operations as may serve a particular implementation. For example, system 400 may perform a first operation to limit which portion of the scan images is considered when processing the post-operative scan images, a second operation to estimate an electrode path of the electrode lead within the limited portion of the cochlea, and a third operation to analyze relatively high intensity regions in the post-operative scan images along the estimated electrode path.

At operation 510, system 400 may determine candidate positions of the plurality of electrode contacts in relation to the cochlea. The candidate positions may be determined in any suitable manner. For example, system 400 may identify each region within the post-operative scan images that has an intensity above a predefined threshold intensity as a candidate position.

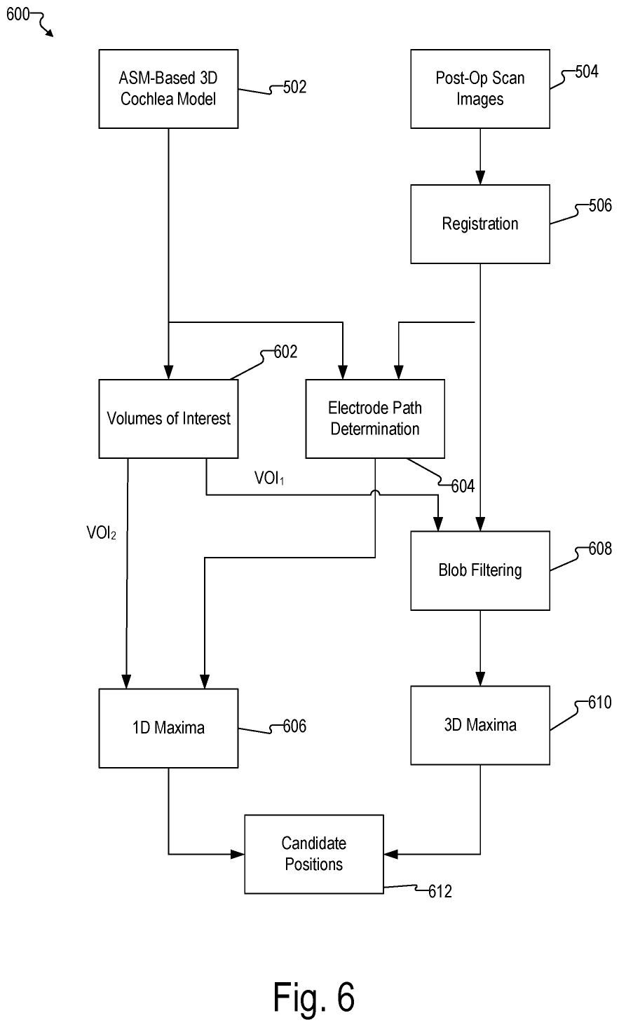

FIG. 6 depicts a flow diagram 600 with exemplary operations that may be performed by system 400 to process the post-operative scan images together with an ASM-based 3D cochlea model. In the example shown in FIG. 6, operations 502-506 may be performed in any suitable manner such as described in relation to FIG. 5.

As shown in FIG. 6, at operation 602, one or more volumes of interest may be determined based on the ASM-based 3D cochlea model. The volumes of interest may define specific regions of the ASM-based 3D cochlea model and/or post-operative scan images that will be subject to processing. System 400 may determine the volumes of interest in any suitable manner. For example, system 400 may use any suitable 3D position and/or orientation information associated with the ASM-based 3D cochlea model and/or post-operative scan images to define the boundaries of the volumes of interest.

System 400 may determine any suitable number of volumes of interest as may serve a particular implementation. For example, system 400 may determine a first volume of interest (VOI₁) and a second volume of interest (VOI₂) for the cochlea. VOI₁ may include a first volume associated with the ASM-based 3D cochlea model and a second volume where electrode contacts included in the plurality of electrode contacts that are not inserted into the cochlea are likely visible. In certain examples, the first volume of VOI₁ may be larger than the ASM-based 3D cochlea model by a predefined margin. The second volume of VOI₁ may have any suitable shape and/or size as may serve a particular implementation. In certain examples, the second volume of VOI₁ may have a conical shape. VOI₂ may include a third volume inside the cochlea. In certain examples, VOI₂ may include image voxels that are inside the cochlea in the post-operative scan images.

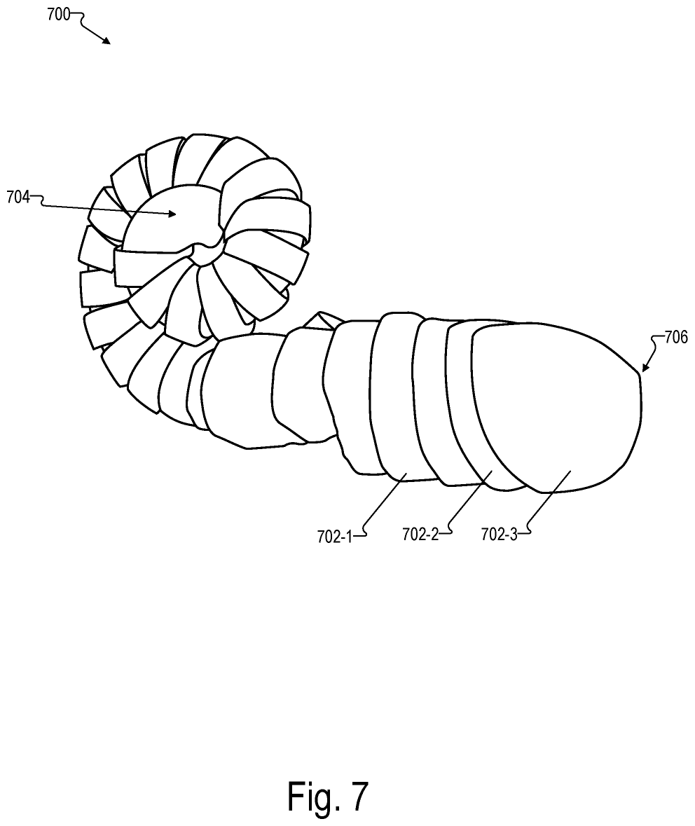

At operation 604, system 400 may determine an estimated electrode path of the electrode lead in relation to the cochlea based on the post-operative scan images. This may be accomplished in any suitable manner. For example, system 400 may slice an ASM-based 3D cochlea model into a plurality of segments ordered from the apex of the cochlea to the round window of the cochlea. System 400 may slice the ASM-based 3D cochlea model into any suitable number of segments as may serve a particular implementation. For example, system 400 may slice the ASM-based 3D cochlea model into five segments, ten segments, fifteen segments, twenty segments, or more than twenty segments. In certain examples, each segment may extend a predefined distance along a length of the ASM-based 3D cochlea model. For example, each segment may extend 1.5 mm along a length of the ASM-based 3D cochlea model. In certain alternative examples, at least some segments may extend different lengths along the ASM-based 3D cochlea model and/or may have different shapes. To illustrate, FIG. 7 shows an exemplary depiction 700 of an ASM-based 3D cochlea model that has been sliced according to principles described herein. As shown in FIG. 7, depiction 700 includes a plurality of segments 702 (e.g., segments 702-1, 702-2, 702-3, etc.) that extend from an apex portion 704 of the cochlea to a portion 706 of the cochlea at the round window.

As part of determining the estimated electrode path, system 400 may determine an intensity of regions within each segment included in the plurality of segments to determine whether a potential electrode contact position is located within the segments. This may be accomplished in any suitable manner. For example, system 400 may analyze each segment individually to determine whether each segment includes one or more regions that have an intensity above a predefined threshold intensity. To illustrate, system 400 may determine that a first segment (e.g., segment 702-1) included in the plurality of segments includes a first region has an intensity above a predefined threshold intensity and is a first candidate position for a first electrode contact included in the plurality of electrode contacts. System 400 may further determine that a second segment (e.g., segment 702-2) included in the plurality of segments includes a second region that has an intensity above the predefined threshold intensity and is a second candidate position for a second electrode contact included in the plurality of electrode contacts. System 400 may further determine that a third segment (e.g., segment 702-3) included in the plurality of segments does not include a region that has an intensity above the predefined threshold intensity and as such does not include a candidate position. This process may be repeated for each of the segments included in the plurality of segments. In some examples, each segment may only include one region that has an intensity above the predefined threshold intensity. In certain alternative examples, at least some segments may have either no regions of high intensity or more than one region of intensity above the predefined threshold intensity. System 400 may then connect the regions of high intensity in adjacent segments in any suitable manner to estimate an electrode path of the electrode lead in relation to the cochlea. For example, system 400 may combine information regarding locations of candidate positions, distances between adjacent candidate positions, etc. in a manner that approximates the electrode path of the electrode lead within the cochlea.

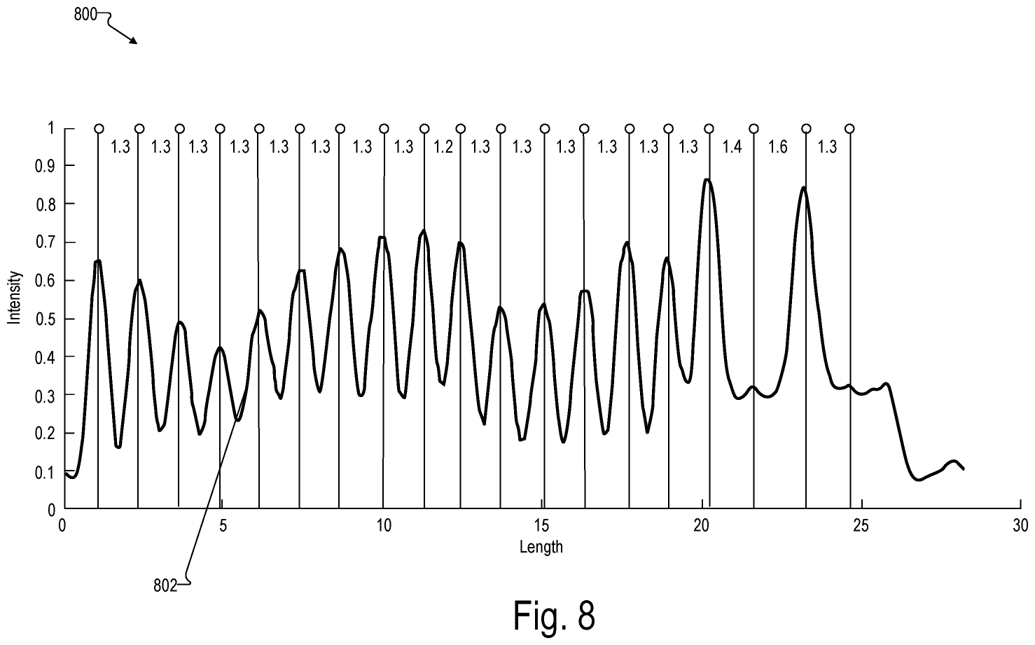

Returning to FIG. 6, at operation 606, system 400 may perform a one-dimensional detection of local maxima along the estimated electrode lead path determined in operation 604. This may be accomplished in any suitable manner. For example, system 400 may plot a graph that shows an intensity detected along the estimated electrode lead path as a function of distance. System 400 may consider any local maxima that is above a predefined threshold intensity as a candidate position for an electrode contact. To illustrate, FIG. 8 shows an exemplary graph 800 that depicts a one-dimensional detection of local maxima where intensity is plotted along the y-axis and length is plotted along the x-axis. In the example shown in FIG. 8, a curve 802 may represent a normalized intensity along the estimated electrode path. The normalized intensity may be obtained by dividing the intensity by a maximal observed value. In the example shown in FIG. 8, the length depicted along the x-axis is in millimeters. As shown in FIG. 8, multiple peaks are depicted along the x-axis. Each peak along the x-axis may correspond to a potential electrode contact position at a certain distance along the electrode lead.

In certain examples, the processing of the post-operative scan images together with the ASM may further include performing an image filter operation on at least one of the first volume of interest or the second volume of interest. Such an image filter operation may emphasize regions of high intensity that may be created by the plurality of electrode contacts in post-operative scan images. The regions of high intensity may be referred to herein as blobs that may be represented by groupings of pixels within the post-operative scan images that are relatively brighter than other regions of the post-operative scan images. Such blobs may represent potential locations of electrode contacts, artifacts caused by the electrode contacts, or anatomical features (e.g., bone structure near the cochlea).

Returning to FIG. 6, at operation 608, system 400 may perform an image filter operation at operation that includes a blob filtering operation. As shown in FIG. 6, system 400 may perform the blob filtering operation with respect to the registered post-operative scan images and with respect to VOI₁. The blob filtering operation may include filtering the registered post-operative scan images in any suitable manner. For example, the blob filtering operation may include a 3D analysis of any relatively high intensity bright spots within VOI₁. In such examples, the 3D analysis may include system 400 performing image filtering based on an eigen decomposition of 3D Hessians applied to VOI₁ to emphasize blobs (e.g., spherical blobs) of high intensities in the post-operative scan images that may be created by metallic electrode contacts of an electrode lead.

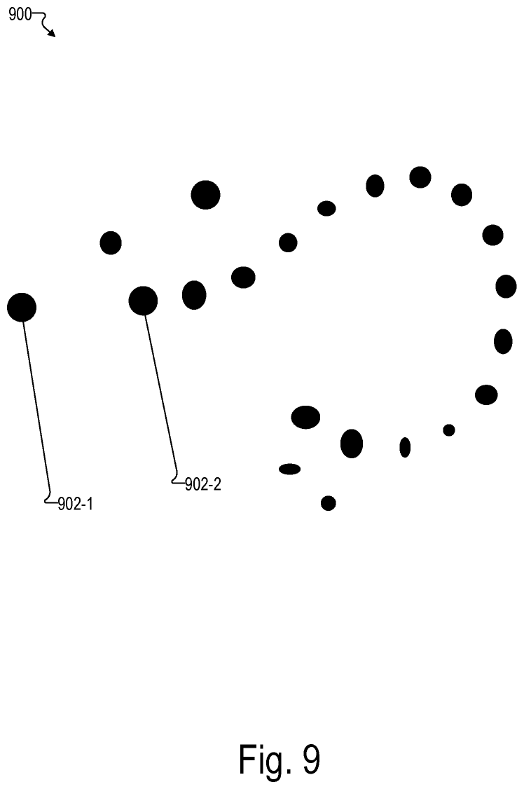

FIG. 9 illustrates an exemplary diagram 900 that shows how a plurality of blobs 902 (e.g., blobs 902-1, 902-2, etc.) may visually appear after a blob filtering operation. Each of the blobs shown in FIG. 9 (including those labeled as 902-1, 902-2, etc. as well as the others) may correspond to a candidate electrode contact position of an electrode lead in relation to the cochlea. Blobs 902 are shown as being black in FIG. 9 for illustrative purposes. However, it is understood that blobs 902 may actually appear as relatively higher intensity bright spots (e.g., white spots) in scan images as compared to other relatively darker regions of the scan images.

Returning to FIG. 6, at operation 610 system 400 may perform a 3D maxima operation with respect to post-operative scan images after the blob filtering performed in operation 606. As shown in FIG. 6, the 3D maxima operation may be performed with respect to VOI₁. In so doing, the 3D maxima operation may result in a set of candidates for the positions of electrode contacts that are inside and/or outside of the cochlea. System 400 may perform the 3D maxima operation in any suitable manner. For example, system 400 may perform a thresholding operation to determine the position(s) of the electrode contact(s) that are inside and/or outside of the cochlea.

At operation 612, system 400 may determine the candidate positions for the plurality of electrode contacts. This may be accomplished in any suitable manner. For example, system 400 may determine a first set of candidates for electrode contact positions that are within the first volume of interest and are outside of the cochlea. The first set of candidates for the electrode contact positions may correspond to candidate positions determined based on the 3D maxima operation performed in operation 610. Further, system 400 may determine a second set of candidates for the electrode contact positions that are within the cochlea in the second volume of interest. The second set of candidates may correspond to the candidate positions determined based on the one-dimensional maxima operation performed at operation 606. System 400 may then combine the first set of candidates and the second set of candidates in any suitable manner to determine the candidate positions at operation 612.

The candidate positions determined at operation 612 may include regions of high intensity that correspond to electrode contacts as well as regions of high intensity caused by artifacts and/or anatomical features. Accordingly, system 400 may perform one or more operations to determine which candidate positions are not likely to be associated with a position of an electrode contact and remove them from consideration. In certain examples, system 400 may implement a graph matching process to facilitate such a determination. Such a graph matching process may include system 400 comparing a noisy graph of the candidate positions to an expected graph of the electrode contacts and a plurality of potential graphs of the electrode contacts. To that end, system 400 may generate a noisy graph based on the candidate positions of the plurality of electrode lead and based on electrode lead information. The noisy graph may be configured in any suitable manner. In certain examples, the noisy graph may include a plurality of nodes with each node included in the plurality of nodes corresponding to one of the candidate positions for the electrode contacts. The noisy graph may further include edges that connect adjacent nodes included in the plurality of nodes. A distance between adjacent nodes may correspond to a distance between adjacent candidate positions.

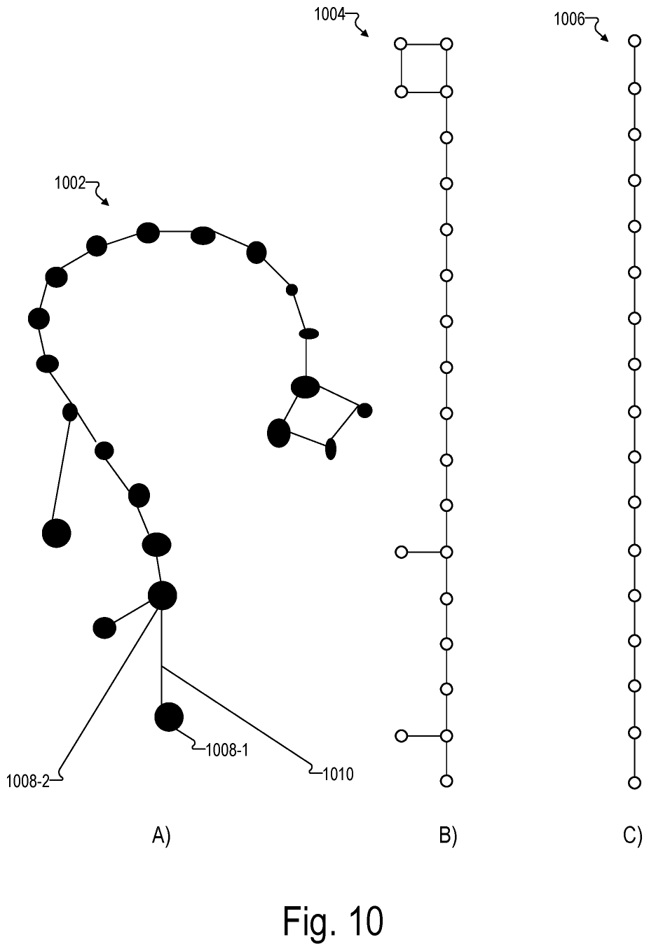

FIGS. 10A-10C show exemplary graphs that may be used during a graph matching process such as described herein. As shown in FIGS. 10A-10C, each graph includes a plurality of nodes and a plurality of edges connecting the nodes. For example, a graph 1002 shown in FIG. 10A includes a first node 1008-1 that corresponds to a first candidate electrode contact position and a second node 1008-2 that corresponds to a second candidate electrode contact position. An edge 1010 connects first node 1008-1 and second node 1008-2 and represents a distance between first node 1008-1 and second node 1008-2. Graph 1002 shown in FIG. 10A depicts an implanted orientation of the candidate electrode contact positions with respect to one another. FIG. 10B depicts a noisy graph 1004 that may be generated based on graph 1002. Graph 1002 is depicted for illustrative purposes to show where noisy graph 1004 may come from. FIG. 10C depicts an expected graph 1006 of the plurality of contacts of an electrode lead where nodes represent electrode contacts and edges represent expected distances between adjacent electrode contacts. As shown in FIG. 10A, graph 1002 and noisy graph 1004 include additional candidate positions and edges that are not included in the expected graph.

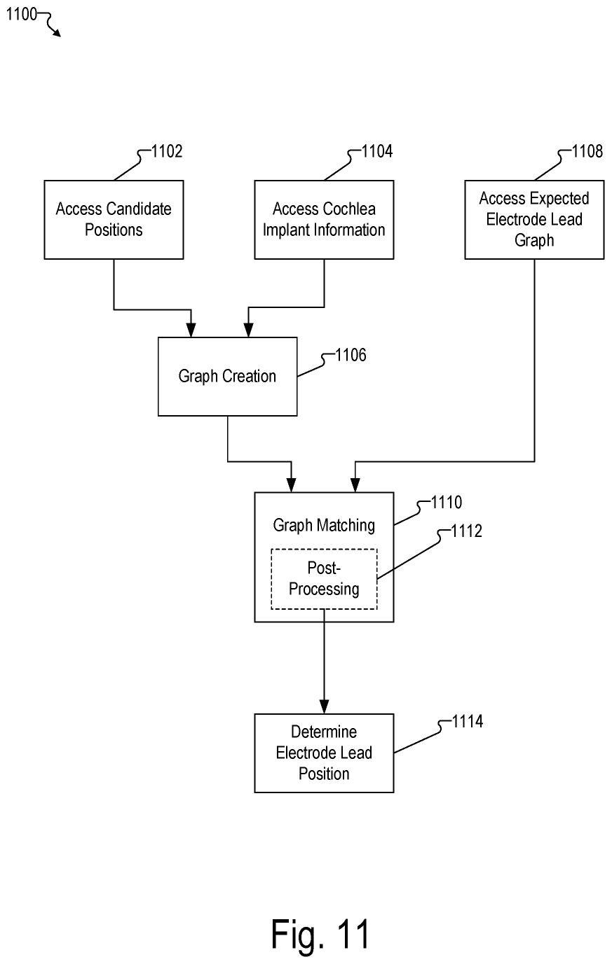

FIG. 11 shows an exemplary flow diagram 1100 of a graph matching process that may be performed by system 400 in certain examples. At operation 1102, system 400 may access candidate positions for the plurality of electrode contacts. System 400 may access the candidate positions in any suitable manner and from any suitable source. For example, system 400 may access the candidate positions determined at operation 612 in FIG. 6. At operation 1104, system 400 may access cochlea implant information such as information defining dimensions (e.g., length, width, etc.) of the electrode lead, an expected distance between electrode contacts, information defining an expected angle between adjacent electrode contacts, the number of electrode contacts on the electrode lead, the orientation of the electrode contacts on the electrode lead, and/or any other suitable information.

At operation 1106, system 400 may generate a noisy graph of the candidate positions. This may be accomplished in any suitable manner. For example, system 400 may identify candidate positions as nodes and define edges distances between the nodes to result in a noisy graph such as noisy graph 1004 shown in FIG. 10B. The edges may connect two nodes if their distance corresponds to an expected difference (with some tolerance) between electrode contacts.

At operation 1108, system 400 may access an expected electrode lead graph such as expected electrode lead graph 1006 shown in FIG. 10C. The expected electrode lead graph may depict expected positions of electrode contacts along a length of an electrode lead at expected distances from one another.

At operation 1110, system 400 may perform a graph matching process in which the noisy graph may be compared to an expected electrode lead graph and a plurality of potential graphs of the plurality of electrode contacts. Each potential graph included in the plurality of potential graphs may represent a different possible configuration of the potential candidate positions represented in the noisy graph. The potential graphs may be limited to ones that include an expected number of nodes and that include edges that substantially approximate the expected intra-contact distances. For example, an electrode lead may have sixteen contacts so the potential graphs may be limited to those with 16 nodes. In certain examples, system 400 may generate the plurality of potential graphs. In such examples, system 400 may use any suitable machine learning algorithm to determine the different potential graphs from the noisy graph. System 400 may then select the potential graph that best matches the expected electrode lead graph. System 400 may use any suitable criteria to determine which of the plurality of potential graphs is substantially a match to the expected electrode lead graph. For example, system 400 may assign similarity scores to each of the graphs included in the plurality of potential graphs. A graph with the highest similarity score may be considered as substantially a match to the expected graph.

In certain examples, the graph matching process may include a fast graph matching process that takes into consideration different distances associated with an electrode lead of a cochlear implant system. For example, an inter-contact distance d_{c} and a distance to the reference contact dᵣ are two relevant distance values may be found in electrode contacts of a cochlear implant system. In such examples, system 400 may use d_{c} and dᵣ as edge weights for the plurality of potential graphs. The noisy graph is expected to include, as a subgraph, a weighted representation of the expected graph of the electrode lead. System 400 may apply a fast graph matching process to search for the expected electrode lead graph from a plurality of different weighted subgraphs of the noisy graph.

In certain examples, at operation 1112, system 400 may perform one or more post-processing operations to limit the number of potential graphs to be considered during graph matching. The box representing operation 1112 is shown in dashed lines because operation 1112 may be optionally performed by system 400. In certain examples, such post processing operations may include system 400 excluding graphs from the plurality of potential graphs based on an attribute of a potential graph deviating from an expected attribute that would be expected in an implanted electrode lead. For example, an angle between adjacent electrode contacts may be expected to be below a predefined angle in an implanted electrode lead. Accordingly, in such examples, system 400 may exclude any potential graphs where an angle between consecutive edges is above a predefined threshold angle.

At operation 1114, system 400 may determine the position and orientation of the electrode lead in relation to the cochlea including the position of each electrode contact included in the plurality of electrode contacts. This may be accomplished in any suitable manner. For example, system 400 may conform the potential graph selected at operation 1106 to the implanted electrode lead path. System 400 may exclude any candidate positions that do not match with nodes on the potential graph selected at operation 1106.

System 400 may use the positions of the electrode contacts determined at operation 1114 in any suitable manner. For example, the positions of the electrode contacts may be used for optimization purposes, for diagnostics purposes, and/or for any other suitable purpose. For example, system 400 may determine, based on the positions of the electrode contacts, that the electrode lead is positioned correctly within the cochlea. Alternatively, system 400 may determine, based on the positions of the electrode contacts, that the electrode lead is not inserted sufficiently into the cochlea (e.g., one or more electrode contacts are positioned outside of the cochlea), which may negatively affect operation of the cochlear implant system. Additionally or alternatively, based on the positions of the electrode contacts, system 400 may be able to determine whether the electrode lead has caused damage to the cochlea by, for example, translocating the basilar membrane. Additionally or alternatively, system 400 may use the positions of the electrode contacts to select which electrode contact to use for stimulation based on their respective position with respect to the tonotopically arranged cochlea.

In certain examples, system 400 may use the positions of the electrode contacts determined at operation 1214 to generate a 3D model of the electrode lead in relation to the cochlea. The 3D model of the electrode lead may depict the position, orientation, etc. of the implanted electrode lead in 3D in relation to the cochlea.

In certain examples, system 400 may be configured to provide a representation of the determined positions of electrode contacts for display to a user. Such a representation may be provided in any suitable manner. For example, the representation may include a reconstruction of the electrode lead (e.g., a 3D model of the electrode lead) and the contacts superimposed over a 3D representation of the cochlea. In certain examples, the representation may correspond to a computer-aided design (CAD) 3D rendering of the electrode lead displayed together with scan images of the cochlea.

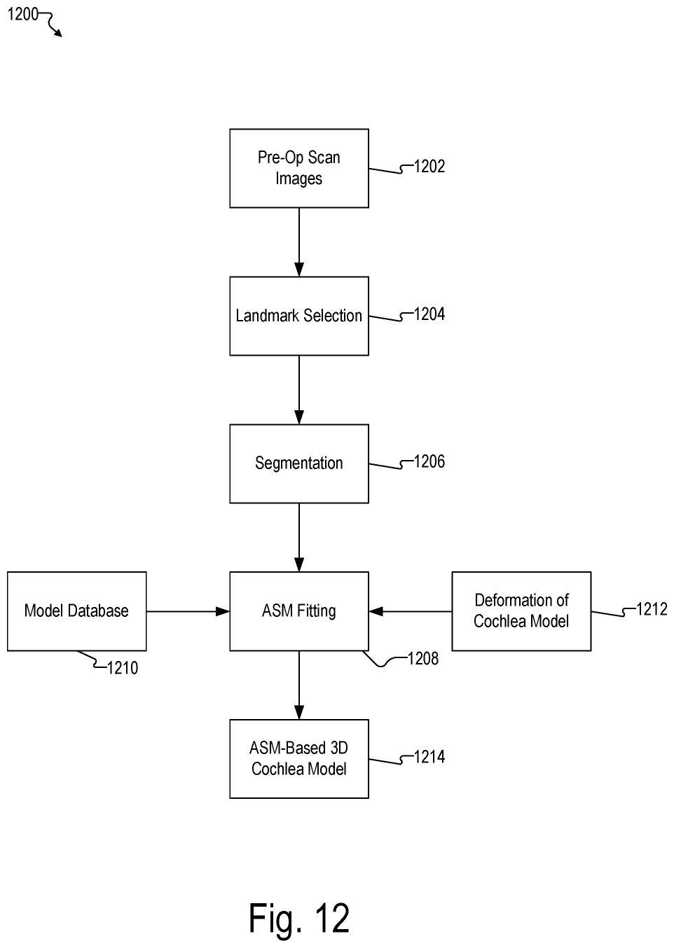

In certain examples, system 400 may generate an ASM-based cochlea model based on pre-operative scan images of the cochlea. In so doing, system 400 may estimate the shape and/or position of the scala tympani, scala vestibuli/scala media, and basilar membrane/osseous spiral lamina from the pre-operative scan images. FIG. 12 illustrates an exemplary flow diagram 1200 that includes various operations that may be performed by system 400 in generating an ASM-based 3D cochlea model. As shown in FIG. 12, at operation 1202, system 400 may access or otherwise obtain pre-operative scan images of the cochlea that depict the cochlea prior to implantation of the electrode lead in the cochlea.

At operation 1204, system 400 may select landmarks for the cochlea within the post-operative scan images. System 400 may select any suitable landmarks as may serve a particular implementation. In certain examples, the landmarks may be manually selected by, for example, a user such as a clinician. In certain alternative examples, the landmarks may be automatically selected by system 400 without requiring input from a user. For example, system 400 may implement any suitable machine learning algorithm or neural network algorithm to select the landmarks in certain implementations.

At operation 1206, system 400 may perform a segmentation process. Such a segmentation process may be performed in any suitable manner. For example, system 400 may use a threshold based segmentation that includes considering sharp transitions of hounsfield units. A hounsfield threshold may be selected by considering an area around the landmarks to provide good segmentation independent of the hounsfield range of the scan images.

At operation 1208, system 400 may fit the ASM to the shape segmented at operation 1206. An ASM of the cochlea wall, the basilar membrane, and the osseous spiral lamina may be used at operation 1208. The basilar membrane and osseous spiral lamina may be represented by connecting two points, in each cross section, on the outer wall. The first point may be a projection of the spiral ligament on the lateral wall, the second point may be the intersection between the osseous spiral lamina and the medial wall. In this way, as the shape of the outer wall changes, so does the location of those two points and consequently the estimate of the basilar membrane and the osseous spiral lamina.

In certain examples, system 400 may use an ASM that is previously built based on a plurality of cochlea models segmented from high resolution scan images (e.g., high resolution CT scan images). As shown, system 400 may leverage a cochlea model database at operation 1210 to facilitate fitting the ASM at operation 1208. Such a cochlea model database may include information regarding a plurality of cochlea models that have been previously generated for other users. Cochlea models in the cochlea model database may be described as a combination of the scala tympani, the scala vestibuli, and the basilar membrane/osseous spiral lamina, and as an open duct. The cochlea models may be described till about the 600° depth of the cochlea. This may facilitate higher precision in the basal turn. The apical end of the cochlea may not be relevant for the cochlea implant and may complicate the fitting process. As such, the apical end of the cochlea may not be considered in certain examples when fitting the ASM.

System 400 may fit the ASM to the segmented shape in any suitable manner. For example, for the fitting, the outer wall of the segmented cochlea as well as the selected landmarks may be used. Thus, the fitting process may correspond to a point and surface-based process. Coherent point drifting (CPD), gaussian process regression (GPR) and regularized Laplacian deformation may be used to fit the ASM and select the right variations of shapes to lower the difference from segmentation to the ASM.

In certain examples, system 400 may deform a cochlea model at operation 1212 to facilitate fitting the ASM at operation 1208. This may be accomplished in any suitable manner. For example, system 400 may use regularized Laplacian deformation in certain examples. The deformation of the cochlea model may result in an estimation of the anatomical structure of the cochlea for the recipient.

At operation 1214, system 400 may output an ASM-based 3D cochlea model, which may be used in any suitable manner described herein to determine the positions of electrode contacts with respect to the cochlea.

Although flow diagram 1200 shows a process where an ASM-based 3D cochlea model is generated based on pre-operative scan images, it is understood that ASM-based 3D cochlea models may be generated in other ways in certain alternative implementations. For example, in certain alternative implementations, system 400 may generate an ASM-based 3D cochlea model based only on post-operative scan images. In such examples, system 400 may perform operations similar to those depicted in FIG. 12 with respect to the post-operative scan images to generate an ASM-based 3D cochlea model.

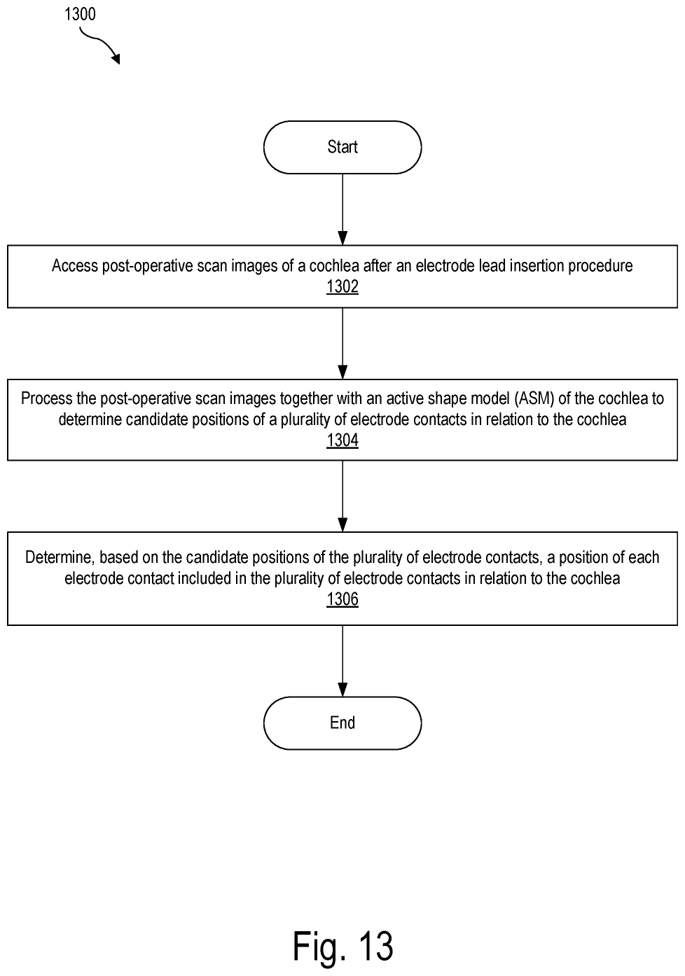

FIG. 13 illustrates an exemplary method 1300 for determining a position of an electrode lead within a cochlea. While FIG. 13 illustrates exemplary operations according to one embodiment, other embodiments may omit, add to, reorder, and/or modify any of the operations shown in FIG. 13.

At operation 1302, an electrode lead position detection system (e.g., electrode lead position detection system 400) may access post-operative scan images of a cochlea after an electrode lead insertion procedure. Operation 1302 may be performed in any of the ways described herein.

At operation 1304, the electrode lead position detection system may process the post-operative scan images together with an active shape model (ASM) of the cochlea to determine candidate positions of a plurality of electrode contacts in relation to the cochlea. Operation 1304 may be performed in any of the ways described herein.

At operation 1306, the electrode lead position detection system may determine, based on the candidate positions of the plurality of electrode contacts, a position of each electrode contact included in the plurality of electrode contacts in relation to the cochlea. Operation 1306 may be performed in any of the ways described herein.

In some examples, a computer program product embodied in a non-transitory computer-readable storage medium may be provided. In such examples, the non-transitory computer-readable storage medium may store computer-readable instructions in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

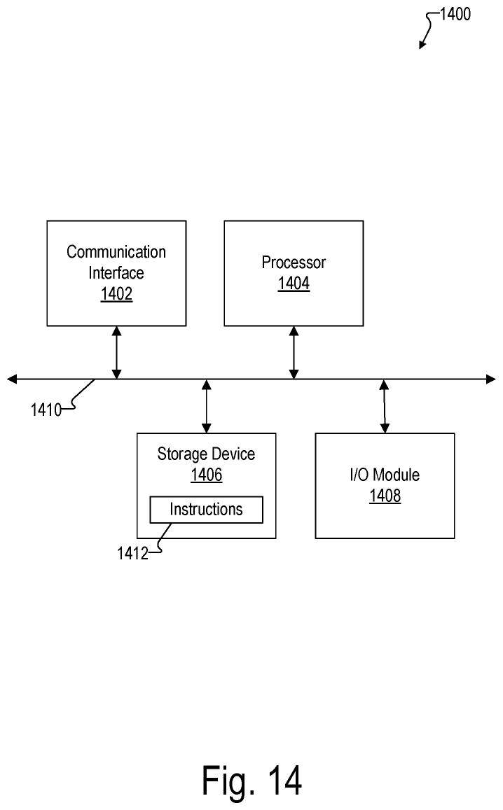

FIG. 14 illustrates an exemplary computing device 1400 that may be specifically configured to perform one or more of the processes described herein. As shown in FIG. 14, computing device 1400 may include a communication interface 1402, a processor 1404, a storage device 1406, and an input/output (“I/O”) module 1408 communicatively connected one to another via a communication infrastructure 1410. While an exemplary computing device 1400 is shown in FIG. 14, the components illustrated in FIG. 14 are not intended to be limiting. Additional or alternative components may be used in other embodiments. Components of computing device 1400 shown in FIG. 14 will now be described in additional detail.

Communication interface 1402 may be configured to communicate with one or more computing devices. Examples of communication interface 1402 include, without limitation, a wired network interface (such as a network interface card), a wireless network interface (such as a wireless network interface card), a modem, an audio/video connection, and any other suitable interface.

Processor 1404 generally represents any type or form of processing unit capable of processing data and/or interpreting, executing, and/or directing execution of one or more of the instructions, processes, and/or operations described herein. Processor 1404 may perform operations by executing computer-executable instructions 1412 (e.g., an application, software, code, and/or other executable data instance) stored in storage device 1406.

Storage device 1406 may include one or more data storage media, devices, or configurations and may employ any type, form, and combination of data storage media and/or device. For example, storage device 1406 may include, but is not limited to, any combination of the non-volatile media and/or volatile media described herein. Electronic data, including data described herein, may be temporarily and/or permanently stored in storage device 1406. For example, data representative of computer-executable instructions 1412 configured to direct processor 1404 to perform any of the operations described herein may be stored within storage device 1406. In some examples, data may be arranged in one or more databases residing within storage device 1406.

I/O module 1408 may include one or more I/O modules configured to receive user input and provide user output. I/O module 1408 may include any hardware, firmware, software, or combination thereof supportive of input and output capabilities. For example, I/O module 1408 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1408 is configured to provide graphical data to a display for presentation to a user. The graphical data may be representative of one or more graphical user interfaces and/or any other graphical content as may serve a particular implementation.

In some examples, any of the systems, hearing devices, computing devices, and/or other components described herein may be implemented by computing device 1400. For example, memory 402 may be implemented by storage device 1406, and processor 404 may be implemented by processor 1404.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory that stores instructions; and
a processor communicatively coupled to the memory and configured to execute the instructions to perform a process comprising:
accessing post-operative scan images of a cochlea after an electrode lead insertion procedure, the post-operative scan images depicting an electrode lead with a plurality of electrode contacts inserted at least partially within the cochlea;
processing the post-operative scan images together with an active shape model (ASM) of the cochlea to determine candidate positions of the plurality of electrode contacts in relation to the cochlea; and
determining, based on the candidate positions of the plurality of electrode contacts, a position of each electrode contact included in the plurality of electrode contacts in relation to the cochlea, the determining of the position of each electrode contact comprising:
generating, based on the candidate positions of the plurality of electrode contacts and electrode lead information, a noisy graph of the candidate positions of the plurality of electrode contacts; and
selecting, based on a comparison of the noisy graph to an expected graph of the plurality of electrode contacts, an estimated graph of the plurality of electrode contacts from a plurality of potential graphs of the plurality of electrode contacts.

2. The system of claim 1, wherein the process further comprises registering the post-operative scan images with pre-operative scan images of the cochlea.

3. The system of claim 1, wherein the processing of the post-operative scan images together with the ASM comprises determining an estimated electrode path of the electrode lead in relation to the cochlea based on the post-operative scan images.

4. The system of claim 3, wherein the determining of the estimated electrode path comprises:
slicing an ASM-based three-dimensional (3D) cochlea model into a plurality of segments ordered from an apex of the cochlea to a round window of the cochlea;
determining that a first segment included in the plurality of segments includes a first region that has an intensity above a predefined threshold intensity and is a first candidate position for a first electrode contact included in the plurality of electrode contacts; and
determining that a second segment included in the plurality of segments includes a second region that has an intensity above the predefined threshold intensity and is a second candidate position for a second electrode contact included in the plurality of electrode contacts.

5. The system of claim 1, wherein the processing of the post-operative scan images together with the ASM comprises determining the candidate positions of the plurality of electrode contacts in relation to the cochlea based on an ASM-based three-dimensional (3D) cochlea model generated based on pre-operative scan images.

6. The system of claim 5, wherein:
the processing of the post-operative scan images together with the ASM further comprises determining a first volume of interest and a second volume of interest;
the first volume of interest includes a first volume associated with the ASM-based 3D cochlea model and a second volume where electrode contacts included in the plurality of electrode contacts that are not inserted into the cochlea are likely visible; and
the second volume of interest includes a third volume inside the cochlea.

7. The system of claim 6, wherein the processing of the post-operative scan images together with the ASM further includes performing an image filter operation on at least one of the first volume of interest or the second volume of interest to emphasize regions of high intensity created by the plurality of electrode contacts in the post-operative scan images.

8. The system of claim 6, wherein:
the processing of the post-operative scan images together with the ASM further includes:
determining a first set of candidates for electrode contact positions that are within the first volume of interest and are at least one of inside of the cochlea or outside of the cochlea; and
determining a second set of candidates for electrode contact positions within the cochlea in the second volume of interest; and
the first set of candidates and the second set of candidates are combined to determine the candidate positions.

9. The system of claim 8, wherein the determining of the second set of candidates includes performing a one-dimensional detection of local maxima along an estimated electrode lead path within the second volume of interest.

10. (canceled)

11. The system of claim 1, wherein the noisy graph includes:
a plurality of nodes with each node included in the plurality of nodes corresponding to one of the candidate positions; and
edges that connect adjacent nodes included in the plurality of nodes where a distance between the adjacent nodes corresponds to a distance between adjacent candidate positions.

12. The system of claim 11, wherein the selecting of the estimated graph includes excluding graphs from the plurality of potential graphs based on an angle between consecutive edges being above a predefined threshold.

13. The system of claim 1, wherein the post-operative scan images correspond to computerized tomography (CT) scan images.

14. The system of claim 1, wherein the process further comprises determining a surface of a wall of the cochlea based on a thresholding-based segmentation process.

15. The system of claim 14, wherein the process further comprises deforming the ASM to fit to the surface of the wall of the cochlea to generate an ASM-based three-dimensional (3D) cochlea model.

16. The system of claim 15, wherein:
the determining of the surface of the wall of the cochlea is performed with respect to pre-operative scan images of the cochlea; and
the ASM is deformed to fit the surface of the wall of the cochlea based on the pre-operative scan images.

17. A computer program product embodied in a non-transitory computer readable storage medium and comprising computer instructions for performing a process comprising:
accessing post-operative scan images of a cochlea after an electrode lead insertion procedure, the post-operative scan images depicting an electrode lead with a plurality of electrode contacts inserted at least partially within the cochlea;
processing the post-operative scan images together with an active shape model (ASM) of the cochlea to determine candidate positions of the plurality of electrode contacts in relation to the cochlea; and
determining, based on the candidate positions of the plurality of electrode contacts, a position of each electrode contact included in the plurality of electrode contacts in relation to the cochlea, the determining of the position of each electrode contact comprising:
generating, based on the candidate positions of the plurality of electrode contacts and electrode lead information, a noisy graph of the candidate positions of the plurality of electrode contacts; and
selecting, based on a comparison of the noisy graph to an expected graph of the plurality of electrode contacts, an estimated graph of the plurality of electrode contacts from a plurality of potential graphs of the plurality of electrode contacts.

18. (canceled)

19. The computer program product of claim 17, wherein the noisy graph includes:
a plurality of nodes with each node included in the plurality of nodes corresponding to one of the candidate positions; and
edges that connect adjacent nodes included in the plurality of nodes where a distance between the adjacent nodes corresponds to an expected distance between adjacent electrode contacts.

20. A method comprising:
accessing, by an electrode lead position detection system, post-operative scan images of a cochlea after an electrode lead insertion procedure, the post-operative scan images depicting an electrode lead with a plurality of electrode contacts inserted at least partially within the cochlea;
processing, by the electrode lead position detection system, the post-operative scan images together with an active shape model (ASM) of the cochlea to determine candidate positions of the plurality of electrode contacts in relation to the cochlea; and
determining, by the electrode lead position detection system, based on the candidate positions of the plurality of electrode contacts, a position of each electrode contact included in the plurality of electrode contacts in relation to the cochlea, the determining of the position of each electrode contact comprising:
generating, based on the candidate positions of the plurality of electrode contacts and electrode lead information, a noisy graph of the candidate positions of the plurality of electrode contacts; and
selecting, based on a comparison of the noisy graph to an expected graph of the plurality of electrode contacts, an estimated graph of the plurality of electrode contacts from a plurality of potential graphs of the plurality of electrode contacts.

21. The method of claim 20, wherein the noisy graph includes:
a plurality of nodes with each node included in the plurality of nodes corresponding to one of the candidate positions; and
edges that connect adjacent nodes included in the plurality of nodes where a distance between the adjacent nodes corresponds to a distance between adjacent candidate positions.

22. The method of claim 20, wherein the processing of the post-operative scan images together with the ASM comprises determining an estimated electrode path of the electrode lead in relation to the cochlea based on the post-operative scan images.
